(19) United States
(12) Patent Application Publication
(10) Pub. No.: US 2005/0181055 A1
(43) Pub. Date: Aug. 18, 2005

(54) Pharmaceutical compositions of quinapril

(76) Inventors: Rajeev Shanker Mathur, Gurgaon (IN); Sameer Manan, Delhi (IN); Sanjeev Sethi, Faridabad (IN)

Correspondence Address: Jay R. Deshmukh, Esq.;Ranbaxy Inc., Suite 2100, 600 College Road East, Princeton, NJ, 08540, US

(21) Appl. No.: 10/962,100

(22) Filed: Oct. 8, 2004

(30) Foreign Application Priority Data
Oct. 8, 2003 (IN) 1246/DEL/2003

(51) Int. Cl. A61K 9/14; A61K 33/10; A61K 31/401

(52) U.S. Cl. 424/486; 514/423; 424/686

(57) ABSTRACT

The present invention relates to pharmaceutical compositions of quinapril and processes for their preparation. One pharmaceutical composition includes a therapeutically effective amount of quinapril or an acid addition salt thereof and between about 15% w/w to about 40% w/w of an alkaline substance.

### FIELD OF THE INVENTION

The technical field of the present invention relates to pharmaceutical compositions of quinapril and processes for their preparation.

### BACKGROUND OF THE INVENTION

Quinapril is the ethyl ester of a nonsulfhydryl, angiotensin-converting enzyme (ACE) inhibitor and quinaprilat. It is widely indicated for the treatment of hypertension, heart failure and angioedema. Although the principal mechanism of its antihypertensive effect is thought to be through the renin-angiotensin-aldosterone system, quinapril exerts antihypertensive actions even in patients with low renin hypertension.

Quinapril suffers from instability which is more pronounced when formulated into pharmaceutical dosage forms. Decomposition takes place mainly through cyclization via internal nucleophilic attack to form substituted diketopiperazines, hydrolysis to form diacids, and oxidation leading to discoloration. Regardless of manner, decomposition is undesirable as it results in decreased effectiveness and even toxicity in some cases. Hence, proper selection of the pharmaceutically inert excipients as well as their concentrations is of utmost importance in stabilizing quinapril in pharmaceutical compositions. Various approaches have been proposed and used to stabilize pharmaceutical compositions of quinapril.

U.S. Pat. No. 4,743,450 discloses the use of an effective amount of alkali or alkaline earth metal salts and saccharides as stabilizers against cyclization and hydrolysis respectively. The effective amount of the stabilizer component of the composition (e.g., alkali or alkaline earth metal salts and saccharide) is disclosed as varying from as low as 1% w/w to as high as 90% w/w. All of the examples disclosed in the patent that provide satisfactory stabilization use great amounts (approximately 46% to approximately 88%) of magnesium carbonate in combination with saccharide as stabilizers. Hence, it appears the pharmaceutical compositions comprising quinapril can be stabilized only with the use of a combination of a great amount of alkaline substance and saccharides.

While magnesium carbonate acts as an effective stabilizer of quinapril, its use in pharmaceutical formulations in substantially higher quantities presents certain disadvantages in that magnesium carbonate, being a bulky powder, may be difficult to formulate into a dosage form because of its poor compressibility, moldability and flowability.

Hence, there exists a need for stabilizing quinapril in pharmaceutical compositions using a comparatively low amount of alkaline substance that would not affect processing of the dosage forms.

We have now developed a stable pharmaceutical composition of quinapril or its acid addition salt that has low amounts of an alkaline substance or substances to inhibit cyclization. The composition may further contain one or more polymers of the vinylpyrrolidone moiety to inhibit hydrolysis. Alternatively, we have also developed a saccharide-free stable pharmaceutical composition of quinapril or its acid addition salt which has alkaline substance to inhibit cyclization and one or more polymer of the vinylpyrrolidone moiety to inhibit hydrolysis.

### SUMMARY OF THE INVENTION

In one general aspect there is provided a pharmaceutical composition that includes a therapeutically effective amount of quinapril or an acid addition salt thereof and between about 15% w/w to about 40% w/w of an alkaline substance.

Embodiments of the composition may include one or more of the following features. For example, the pharmaceutical composition may further include one or more polymers of vinylpyrrolidone moiety. The one or more polymers of vinylpyrrolidone moiety may be one or more of povidone, crospovidone, copolyvidone and mixtures thereof. The one or more polymers of vinylpyrrolidone moiety may be present at from about 2% w/w to about 30% w/w of the total composition. In particular, the amount of one or more polymers of vinylpyrrolidone moiety may be present at from about 4% w/w to about 15% w/w of the total composition.

The acid addition salts of quinapril may be one or more of hydrochloride, hydrobromide, orthophosphate, benzoate, maleate, tartrate, succinate, citrate, alicylate, and acetate. In particular, the acid addition salt of quinapril may be quinapril hydrochloride.

The alkaline substance may be salts, hydroxides and oxides of metals. The salts may be one or both of carbonates and bicarbonates. The oxides of metals may be one or more of magnesium, calcium and sodium. The alkaline substance may be magnesium carbonate. The amount of magnesium carbonate may be present at from about 20% w/w to about 30% w/w of the total composition.

The pharmaceutical composition may further include one or more pharmaceutically acceptable inert excipients. The pharmaceutically acceptable inert excipient may be one or more of diluents, binders, disintegrants, coloring agents, flavoring agents or lubricants/glidants.

The pharmaceutical composition may be in the form of one or more of a powder, tablet, granule, pellet, spheroid, caplet or capsule. The composition may be coated with a functional and/or non-functional film forming polymer.

The pharmaceutical composition may have a chemical stability of quinapril against degradation due to cyclization, hydrolysis and oxidation that is not more than about 6% w/w related substances formed on storage at 40±2° C. and 75±5% relative humidity for a period of 6 months. In particular, the chemical stability may be not more than about 3.6% w/w of related substances formed under these conditions.

In another general aspect there is provided a process for the preparation of a pharmaceutical composition. The process includes the steps of: (a) blending together a therapeutically effective amount of quinapril or an acid addition salt thereof and about 15% to about 40% w/w of an alkaline substance to form a mixture; (b) blending the mixture with one or more pharmaceutically inert excipients; and (c) processing into a suitable solid dosage form.

Embodiments of the process may include one or more of the following features or those described above. For example, the blend of step (a) may be granulated. The granulation may be carried out by a wet granulation or a dry granulation technique. In particular, the granulation may be the wet granulation technique and the wet granulation may be carried out using a granulating fluid comprising one or more of methylene chloride, isopropyl alcohol, acetone, methanol, ethanol, water or combinations thereof. The granulating fluid may further include one or more polymers of vinyl pyrrolidone moiety.

The blend of step (a) may be further processed by extrusion-spheronization. The blend of step (a) may further comprises one or more polymers of vinylpyrrolidone moiety.

The blend of step (b) may further comprises one or more polymers of vinylpyrrolidone moiety. In another general aspect there is provided a pharmaceutical composition that includes a therapeutically effective amount of quinapril or an acid addition salt thereof, one or more alkaline substances, and one or more polymers of vinylpyrrolidone moiety.

Embodiments of the composition may include one or more of the following features or those described above. For example, the one or more polymers of vinylpyrrolidone moiety may be povidone, crospovidone or a mixture thereof. The alkaline substances may be about 15% w/w to about 40% w/w of the total composition.

In another general aspect there is provided a method of treating hypertension in a subject in need thereof. The method includes administering a pharmaceutical composition that includes a therapeutically effective amount of quinapril or an acid addition salt thereof and about 15% to about 40% w/w of an alkaline substance. Embodiments of the method may include any of the features described above.

In another general aspect there is provided a method of treating hypertension in a subject in need thereof. The method includes administering a pharmaceutical composition that includes a therapeutically effective amount of quinapril or an acid addition salt thereof, one or more alkaline substances, and one or more polymers of vinylpyrrolidone moiety. Embodiments of the method may include any of the features described above.

The details of one or more embodiments of the inventions are set forth in the description below. Other features, objects and advantages of the inventions will be apparent from the description and claims.

### DETAILED DESCRIPTION OF THE INVENTION

The pharmaceutical compositions of the present invention use a low amount of alkaline substances as stabilizers, yet provide protection for quinapril against degradation by cyclization. The stability may be synergistically enhanced with the incorporation of one or more polymers of the vinylpryrrolidone moiety, as they stabilize the formulation against hydrolysis. These polymers may also perform other functions, such as acting as binding agents, disintegrants, and/or diluents.

The term “stable pharmaceutical composition” as used herein refers to the chemical stability of quinapril against degradation due to cyclization, hydrolysis and oxidation wherein not more than about 6% w/w, and in particular not more than about 4% w/w, of related substances are formed on storage at 40±2° C. and 75±5% relative humidity for a period of 6 months.

The term “quinapril” as used herein includes quinapril and pharmaceutically acceptable salts thereof. Suitable pharmaceutically acceptable salts include one or more of hydrochloride, hydrobromide, orthophosphate, benzoate, maleate, tartrate, succinate, citrate, salicylate, acetate and the like. For example, quinapril hydrochloride may be used.

The pharmaceutical compositions of quinapril may further include one or more additional drug(s) which do not adversely affect the stability of the compositions. Suitable examples of the one or more additional drugs include diuretics, such as hydrochlorothiazide; antitussives, such as dextromethorphan, dextromethorphan hydrobromide, noscapine, carbetapentane citrate and chlophedianol hydrochloride; anti histamines such as chlorpheniramine maleate, phenindamine tartrate, pyrilamine maleate, doxylamine succinate and phenyltoloxamine citrate; decongestants such as phenylephedrine hydrochloride, phenyl proponalamine hydrochloride, pseudoephedrine hydrochloride and ephedrine; various alkaloids, such as codeine phosphate, codeine sulfate and morphine and mineral supplements such as potassium chloride; and the like.

The term “low amount of an alkaline substance” as used herein refers to the use of one or more alkaline substances in an amount less than about 45% w/w of the total pharmaceutical composition. In particular, the amount of alkaline substance to be used may vary from about 15% w/w to about 40% w/w of the total pharmaceutical composition.

Suitable alkaline substances include one or more of salts, such as carbonates, bicarbonates; hydroxides; and oxides of metals, such as magnesium, calcium and sodium. In particular, magnesium carbonate may be used. Magnesium carbonate can be a basic hydrated magnesium carbonate, a normal hydrated magnesium carbonate or a mixture of the two.

Polymers of the vinylpyrrolidone moiety are widely accepted in pharmaceutical industry for their application as binders, disintegrants and diluents. These well-known polymers are found to be highly effective in stabilizing quinapril in pharmaceutical compositions. Suitable polymers of the vinylpryrrolidone moiety include one or more of polyvinylpyrrolidine (PVP), also known as povidone, cross-linked polyvinylpyrrolidine, also known as crospovidone and, vinylpyrrolidone-vinyl acetate copolymer, also known as copolyvidone. Povidone is a synthetic polymer of linear 1-vinyl-2-pyrrolidone groups and is commercially available under brand names such as Kollidon® and Plasdone®. Specific grades include PVP K-12, PVP K-15, PVP K-17, PVP K-25, PVP K-30, PVP K-60, PVP K-90, and PVP K-120 having approximate molecular weights of 2,500, 8,000, 10,000, 30,000, 50,000, 400,000, 1000,000, and 3,000,000, respectively. Crospovidone is obtained by a polymerization process that produces a physically cross-linked polyvinylpyrrolidone, which is insoluble in water and in all the usual solvents. It is commercially available in various grades, such as, Kollidon CL, Crospovidone M, and Kollidon CL-M. Copolyvidone is formed by the copolymerization of vinylpyrrolidone and vinyl acetate and is commercially available as Kollidon VA-64. The amount of the one or more polymers of vinylpyrrolidone moiety may vary from about 2% w/w to about 30% w/w, in particular about 4% w/w to about 15% w/w.

The term “pharmaceutical composition” as used herein includes solid dosage forms including as a tablet, capsule, caplet, pill, powder, spheroid, granule, and the like.

Also provided herein is a pharmaceutical composition of quinapril which includes a therapeutically effective amount of quinapril or its acid addition salt, 15% w/w to 40% w/w of alkaline substance, one or more polymers of vinylpyrrolidone moiety, and one or more pharmaceutically acceptable inert excipients.

The term “pharmaceutically acceptable inert excipients” as used herein includes all physiologically inert excipients used in the pharmaceutical art of dispensing. For example, this term includes one or more of diluents, binders, disintegrants, coloring agents, flavoring agents and lubricants/glidants.

Suitable diluents include one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch pregelatinized, sucrose, sugar compressible, sugar confectioners and the like and combinations thereof.

Suitable binders include one or more of polyvinyl pyrrolidone, methyl cellulose, hydroxypropyl cellulose, HPMC, gelatin, gum Arabic, ethyl cellulose, polyvinyl alcohol, pullulan, pregelatinized starch, agar, tragacanth, sodium alginate, propylene glycol and the like and combinations thereof.

Suitable disintegrants include one or more of microcrystalline cellulose, croscarmellose sodium, crospovidone, carboxymethyl starch sodium, sodium starch glycollate and the like and combinations thereof.

Suitable lubricants and glidants include one or more of colloidal anhydrous silica, stearic acid, magnesium stearate, calcium stearate, talc, hydrogenated castor oil, sucrose esters of fatty acid, microcrystalline wax, yellow beeswax, white beeswax and the like and combinations thereof.

Suitable coloring agents include any FDA approved color or colors for oral use.

The pharmaceutical compositions of quinapril may be prepared by processes known in the prior art including comminuting, mixing, granulation, melting, sizing, filling, drying, molding, immersing, coating, compressing, extrusion-spheronization, etc.

In one embodiment, the pharmaceutical composition of quinapril hydrochloride may be prepared by: (a) blending together an effective amount of quinapril hydrochloride, 15% w/w to 40% w/w of magnesium carbonate, and one or more pharmaceutically acceptable inert excipients; (b) adding a granulating fluid which includes one or more polymers of the vinylpyrrolidone moiety to form a moist mass; (c) drying and sizing the granules; (d) blending the granules with one or more polymers of the vinylpyrrolidone moiety and one or more pharmaceutically inert excipients; and (e) processing into a suitable dosage form.

In another embodiment, a pharmaceutical composition of quinapril hydrochloride may be prepared by: (a) blending together a therapeutically effective amount of quinapril hydrochloride, 15 to 40% w/w of magnesium carbonate, one or more polymers of the vinylpyrrolidone moiety and one or more pharmaceutically acceptable inert excipients; (b) dry granulating the blend by roller compactor or slugging; (c) sizing the granules; (d) blending the granules with one or more polymer of the vinylpyrrolidone moiety and one or more pharmaceutically inert excipients; and (e) processing into a suitable dosage form.

In another embodiment, a pharmaceutical composition of quinapril hydrochloride may also be prepared by: (a) blending together a therapeutically effective amount of quinapril hydrochloride, 15 to 40% w/w of magnesium carbonate, one or more polymer of the vinylpyrrolidone moiety, and one or more pharmaceutically acceptable inert excipients; and (b) processing into a suitable dosage form.

In still another embodiment, a pharmaceutical composition of quinapril hydrochloride may also be prepared by: (a) blending together a therapeutically effective amount of quinapril hydrochloride, 15 to 40% w/w of magnesium carbonate, one or more polymer of the vinylpyrrolidone moiety, and one or more pharmaceutically acceptable inert excipients; (b) adding a granulating fluid or solution/dispersion of one or more pharmaceutically acceptable inert excipients in the granulating fluid to the blend to form a moist mass; (c) passing the wet mass through an extruder equipped with a screen; (d) spheronizing the extrudate in a spheronizer; (e) drying and sizing the spheroids; and (f) processing the spheroids into a suitable dosage form.

Processing into a suitable dosage form can be done by filling the granules, spheroids, blend, or powder obtained from the above processes into capsules or compressing to form the tablet. The tablets may be further coated with one or more functional and/or non-functional layers comprising film-forming materials. The coating may be accomplished using conventional coating techniques.

Suitable film forming materials include one or more of water soluble coating materials of celluloses, such as hydroxy propyl cellulose, hydroxy propyl methyl cellulose, hydroxyethyl cellulose, and methyl hydroxy ethyl cellulose; enteric coating materials of celluloses, such as hydroxy propyl methyl cellulose phthalate, hydroxy propyl methyl cellulose acetate succinate, carboxy methyl ethyl cellulose and cellulose acetate phthalate; and other enteric film coating materials, such as methacrylic acid copolymer, shellac and the like. The coating operation may be performed using commercially available, ready to coat preparations, such as one of the various grades of Opadry®.

Suitable solvents that can be used as the granulating fluid and for preparing the solution/dispersion of coating substances include one or more of methylene chloride, isopropyl alcohol, acetone, methanol, ethanol, water and the like.

The invention is further illustrated by the following examples, which is for illustrative purposes and should not be construed to limit the scope of the invention.

| | Amount (mg/tab and %) | | | | | | | |
|---|---|---|---|---|---|---|---|---|
| | Example 1 | | Example 2 | | Example 3 | | Example 4 | |
| Ingredients | mg/tab | % | mg/tab | % | mg/tab | % | mg/tab | % |
| Intragranular | | | | | | | | |
| Quinapril | 5.42 | 10.84 | 10.83 | 10.84 | 21.66 | 10.84 | 43.32 | 10.84 |
| hydrochloride* | | | | | | | | |
| Magnesium | 12.5 | 25.00 | 25.00 | 25.00 | 50.00 | 25.00 | 100.00 | 25.00 |
| carbonate | | | | | | | | |
| Microcrystalline | 11.58 | 23.16 | 23.17 | 23.16 | 46.34 | 23.16 | 92.68 | 23.16 |
| cellulose | | | | | | | | |
| Crospovidone | 1.00 | 2.00 | 2.00 | 2.00 | 4.00 | 2.00 | 8.00 | 2.00 |
| Povidone | 2.00 | 4.00 | 4.00 | 4.00 | 8.00 | 4.00 | 16.00 | 4.00 |
| Purified water | q.s | | q.s | | q.s | | q.s | |
| Extragranular | | | | | | | | |
| Microcrystalline | 15.00 | 30.00 | 30.00 | 30.00 | 60.00 | 30.00 | 120.00 | 30.00 |
| cellulose | | | | | | | | |
| Crospovidone | 2.00 | 4.00 | 4.00 | 4.00 | 8.00 | 4.00 | 16.00 | 4.00 |
| Magnesium | 0.50 | 1.00 | 1.00 | 1.00 | 2.00 | 1.00 | 4.00 | 1.00 |
| stearate | | | | | | | | |
| Core Tablet | 50.0 | 100.0 | 100.0 | 100.0 | 200.0 | 100.0 | 400.0 | 100.0 |
| Weight | | | | | | | | |
| Coating | | | | | | | | |
| composition | | | | | | | | |
| Opadry AMB | 2.14 | | 4.29 | | 8.57 | | 17.14 | |
| OY-B-28920 | | | | | | | | |
| Purified water | q.s | | q.s | | q.s | | q.s | |
| Total Tablet | 52.14 | | 104.29 | | 208.57 | | 417.14 | |
| Weight | | | | | | | | |

*Equivalent to quinapril 5 mg, 10 mg, 20 mg, and 40 mg for Examples 1-4, respectively

The following steps were used to prepare the quinapril tablets having the compositions of Examples 1-4.

1. Quinapril hydrochloride was blended with magnesium carbonate.

2. Intragranular microcrystalline cellulose was blended with the blend of step 1.

3. Povidone was dissolved in purified water.

4. The blend of step 2 was granulated with solution of step 3.

5. The above granules were dried in a fluidized bed dryer at 60-65° C. till LOD was between 1.5-4% and then suitably sieved.

6. The extragranular microcrystalline cellulose and crospovidone were blended with the granules of step 5.

7. The mixture of step 6 was blended with magnesium stearate and compressed into tablets.

8. Finally the compressed tablets were coated with a solution of Opadry until reaching a target weight build up of about 3%.

Stability Studies

The tablets of Example 4, as prepared above, were subjected to stability studies both under controlled temperature conditions (25±2° C./60±5% RH) and accelerated conditions (40±2° C./70±5% RH). The tablets were analyzed periodically for related substances using HPLC technique over a period of 6 months. The results of the study are presented below in Tables 1 and 2.

TABLE 1
Controlled Room Temperature (25 ± 2° C./60 ± 5% RH) Stability Data
of Quinapril Tablets of Ex. 4

| | Related substances (% w/w) | | | | | |
|---|---|---|---|---|---|---|
| | | | | Any other | Any other | |
| | | | Propyl analogues of | known | unknown | Total |
| Period | Compound A* | Compound B** | quinapril | impurities | impurities | impurities |
| Initial | 0.265 | 0.318 | 0.126 | ND | 0.028 | 0.796 |
| 3 months | 0.210 | 0.331 | 0.126 | ND | 0.037 | 0.758 |
| 6 months | 0.274 | 0.383 | 0.126 | ND | 0.055 | 0.975 |

ND—not detected
*Compound A is Diketopiperazine derivative
**Compound B is Quinaprilat

TABLE 2
Accelerated (40 ± 2° C./70 ± 5% RH) Stability Data of Quinapril Tablets of Ex. 4

| | Related substances (% w/w) | | | | | |
|---|---|---|---|---|---|---|
| | | | | Any other | Any other | |
| | | | Propyl analogues of | known | unknown | Total |
| Period | Compound A* | Compound B** | quinapril | impurities | impurities | impurities |
| Initial | 0.265 | 0.318 | 0.126 | ND | 0.028 | 0.796 |
| 1 month | 0.358 | 0.714 | 0.126 | ND | 0.053 | 1.325 |
| 2 months | 0.380 | 0.875 | 0.128 | ND | 0.053 | 1.524 |
| 3 months | 0.373 | 1.054 | 0.124 | ND | 0.065 | 1.687 |
| 6 months | 0.367 | 1.870 | 0.146 | ND | 0.096 | 2.882 |

ND—not detected
*Compound A is Diketopiperazine derivative
**Compound B is Quinaprilat

Results of the stability studies as listed above demonstrate (1) the importance of a low amount of magnesium carbonate and (2) the use of polymers of the vinylpyrrolidone moiety in stabilizing pharmaceutical compositions of quinapril or its acid addition salts against cyclization and hydrolysis. As shown in Tables 1 and 2, formulating a quinapril pharmaceutical composition according to the two general constraints described above results in the amount of impurities being within the acceptable USP limit of 3.6% w/w.

While the present invention has been described in terms of its specific embodiments, certain modifications and equivalents will be apparent to those skilled in the art and are included within the scope of the present invention. For example, the formulations of Examples 1-4 can be modified to vary the amount of intragranular and extragranular microcrystalline cellulose. Instead of 23.16% intragranular microcrystalline cellulose and 30% extragranular microcrystalline cellulose as described above, the formulations can have up to about 25% intragranular microcrystalline cellulose and down to about 28.16% extragranular microcrystalline cellulose, or values within the ranges bounded by these amounts. Similarly, the amount of coating can be decreased from 2.14 mg, 4.29 mg, 8.57 mg, and 17.14 mg, to 1.75 mg, 3.5 mg, 7 mg, and 14 mg, respectively for the strength tablets. Again, the coating can be decreased to any value within the ranges bounded by these amounts.

1. A pharmaceutical composition comprising a therapeutically effective amount of quinapril or an acid addition salt thereof and between about 15% w/w to about 40% w/w of an alkaline substance.

2. The pharmaceutical composition according to claim 1, further comprising one or more polymers of vinylpyrrolidone moiety.

3. The pharmaceutical composition according to claim 1, wherein the acid addition salts of quinapril comprise one or more of hydrochloride, hydrobromide, orthophosphate, benzoate, maleate, tartrate, succinate, citrate, alicylate, and acetate.

4. The pharmaceutical composition according to claim 3, wherein the acid addition salt of quinapril comprises quinapril hydrochloride.

5. The pharmaceutical composition according to claim 1, wherein the alkaline substance comprises salts, hydroxides and oxides of metals.

6. The pharmaceutical composition according to claim 5, wherein the salts comprise one or both of carbonates and bicarbonates.

7. The pharmaceutical composition according to claim 5, wherein the oxides of metals comprise one or more of magnesium, calcium and sodium.

8. The pharmaceutical composition according to claim 1, wherein the alkaline substance comprises magnesium carbonate.

9. The pharmaceutical composition according to claim 8, wherein the amount of magnesium carbonate comprises from about 20% w/w to about 30% w/w of the total composition.

10. The pharmaceutical composition according to claim 2, wherein the one or more polymers of vinylpyrrolidone moiety comprise one or more of povidone, crospovidone, copolyvidone and mixtures thereof.

11. The pharmaceutical composition according to claim 2, wherein the one or more polymers of vinylpyrrolidone moiety comprise from about 2% w/w to about 30% w/w of the total composition.

12. The pharmaceutical composition according to claim 11, wherein the amount of one or more polymers of vinylpyrrolidone moiety comprise from about 4% w/w to about 15% w/w of the total composition.

13. The pharmaceutical composition according to claim 1, further comprising one or more pharmaceutically acceptable inert excipients.

14. The pharmaceutical composition according to claim 13, wherein the pharmaceutically acceptable inert excipient comprise one or more of diluents, binders, disintegrants, coloring agents, flavoring agents or lubricants/glidants.

15. The pharmaceutical composition according to claims 1, wherein the pharmaceutical composition comprises one or more of a powder, tablet, granule, pellet, spheroid, caplet or capsule.

16. The pharmaceutical composition according to claim 15, wherein the composition is coated with a functional and/or a non-functional film forming polymer.

17. The pharmaceutical composition according to claim 1, wherein the chemical stability of quinapril against degradation due to cyclization, hydrolysis and oxidation is not more than about 6% w/w related substances formed on storage at 40±2° C. and 75±5% relative humidity for a period of 6 months.

18. The pharmaceutical composition according to claim 17, wherein the chemical stability of quinapril against degradation due to cyclization, hydrolysis and oxidation is not more than about 3.6% w/w related substances formed on storage at 40±2° C. and 75±5% relative humidity for a period of 6 months.

19. A process for the preparation of a pharmaceutical composition, the process comprising the steps of:
(a) blending together a therapeutically effective amount of quinapril or an acid addition salt thereof and about 15% to about 40% w/w of an alkaline substance to form a mixture;
(b) blending the mixture with one or more pharmaceutically inert excipients; and,
(c) processing into a suitable solid dosage form.

20. The process according to claim 19, wherein the blend of step (a) is granulated.

21. The process according to claim 20, wherein the granulation is carried out by a wet granulation or a dry granulation technique.

22. The process according to claim 21, wherein the granulation comprises the wet granulation technique.

23. The process according to claim 22, wherein the wet granulation is carried out using a granulating fluid comprising one or more of methylene chloride, isopropyl alcohol, acetone, methanol, ethanol, water or combinations thereof.

24. The process according to claim 23, wherein the granulating fluid further comprises one or more polymers of vinyl pyrrolidone moiety.

25. The process according to claim 19, wherein the blend of step (a) is further processed by extrusion-spheronization.

26. The process according to claim 19, wherein the blend of step (a) further comprises one or more polymers of vinylpyrrolidone moiety.

27. The process according to claim 19, wherein the blend of step (b) further comprises one or more polymers of vinylpyrrolidone moiety.

28. A pharmaceutical composition comprising a therapeutically effective amount of quinapril or an acid addition salt thereof, one or more alkaline substances, and one or more polymers of vinylpyrrolidone moiety.

29. The pharmaceutical composition according to claim 28, wherein the one or more polymers of vinylpyrrolidone moiety comprise povidone, crospovidone or a mixture thereof.

30. The pharmaceutical composition according to claim 28, wherein the alkaline substances comprise about 15% w/w to about 40% w/w of the total composition.

31. A method of treating hypertension in a subject in need thereof, the method comprising administering a pharmaceutical composition comprising a therapeutically effective amount of quinapril or an acid addition salt thereof and about 15% to about 40% w/w of an alkaline substance.

32. A method of treating hypertension in a subject in need thereof, the method comprising administering a pharmaceutical composition comprising a therapeutically effective amount of quinapril or an acid addition salt thereof, one or more alkaline substances, and one or more polymers of vinylpyrrolidone moiety.
